(19) United States
(12) Patent Application Publication
(10) Pub. No.: US 2011/0306828 A1
(43) Pub. Date: Dec. 15, 2011

(54) PROPULSION-UNIT AND ROBOT PROVIDED WITH SUCH A PROPULSION-UNIT

(75) Inventors: Paulus Breedveld, Gouda (NL); Dimitra Dodou, Delft (NL)

(73) Assignee: Technische Universiteit Delft, Delft (NL)

(21) Appl. No.: 13/162,932

(22) Filed: Jun. 17, 2011

Related U.S. Application Data
(63) Continuation of application No. PCT/NL2009/050752, filed on Dec. 10, 2009.

(30) Foreign Application Priority Data
Dec. 18, 2008 (NL) 2002341

(51) Int. Cl. A61B 1/00; B62D 57/02

(52) U.S. Cl. 600/104; 180/7.1; 901/1

(57) ABSTRACT

A propulsion-unit provided with a body and comprising at least one element having an external adhesive layer for providing frictional contact with an object's surface along which the unit in use moves, wherein the element is a supporting layer that supports said adhesive layer, and wherein drive means are provided for moving the supporting layer and the body with respect to each other.

This application is a continuation application of International Patent Application Serial No. PCT/NL2009/050752, entitled “Propulsion-Unit and Robot Provided with Such a Propulsion-Unit”, to Technische Universiteit Delft, filed on Dec. 10, 2009, and the specification and claims thereof are incorporated herein by reference.

This application is a continuation of Netherlands Patent Application Serial No. 2002341, entitled “Propulsion-Unit and Robot Provided with Such a Propulsion-Unit”, filed on Dec. 18, 2008 and the specifications and claims thereof are incorporated herein by reference.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### COPYRIGHTED MATERIAL

Not Applicable.

### BACKGROUND OF THE INVENTION

1. Field of the Invention (Technical Field)

The invention relates to a propulsion-unit provided with a body and comprising at least one element having an external adhesive layer for providing frictional contact with an object's surface along which the unit in use moves.

Such a propulsion-unit is used for locomotion of an intestine inspection and/or intervention device, which is moveable in a person's colon. Such a propulsion-unit is required to have very moderate dimensions, notably having a maximum diameter of approximately 30 mm or less. The propulsion-unit of the invention may however also show other dimensions (smaller or larger) and may suitably be employed for other purposes or at any other place where propulsion is needed, such as in other confined areas or in spaces that are difficultly accessible such as the digestive system, arteries, cavities in the nose or the brain or other bodily parts of a human or animal. The propulsion-unit of the invention may further be employed in other surroundings than animal or human bodies, such as industrial applications with difficultly accessible spaces such as pipes, tubes or waste-systems as exist in factories or plants for the generation of heat or electricity. Notably heating pipes of boilers are known areas in which it is at times required to carry out inspections for cracks or other adverse situations. Such pipes may pose specific problems due to the many bends that are required to pass in order to carry out a reliable inspection.

In the following description emphasis will be placed on a discussion of the propulsion-unit of the invention with reference to its application as a means to provide locomotion to a colonoscope. The invention is however broader in scope and not restricted to this particular application. Other applications as mentioned above are likewise covered by the instant invention and the appended claims.

2. Description of Related Art

A propulsion-unit in accordance with the preamble is known from the doctoral thesis entitled ‘Colonic locomotion’ by Dimitra Dodou of September 2006, ISBN-10: 90-9020968-9 and ISBN-13: 978-90-9020968-5. This doctoral thesis amongst others investigates the application of muco-adhesives and investigates to manipulate friction and to achieve locomotion of a colonoscopic device for movement through the large intestine. FIG. 2.1 of said thesis shows that a muco-adhesive layer is applied at the outer circumference of a series of cylinders that are interconnected by having these cylinders placed on a connecting tube. The cylinders sequentially provide a high frictional contact with the inner wall of the intestine, which is alternated by implementing a periodic reduction of this high frictional contact. Thus, the cylinders with reduced frictional contact may slide through the intestine, and the series of cylinders may move through the intestine by forming a repetitious peristaltic movement.

The application of a muco-adhesive layer on the out-side of the moving cylinders provides a solution to the problem which is known from other propulsion-units that are applied for movement in the large intestine, employing balloons that are repeatedly inflated and deflated. Such systems employing balloons are for instance known from U.S. Pat. No. 3,895,637; U.S. Pat. No. 4,676,228; U.S. Pat. No. 4,690,131; U.S. Pat. No. 5,337,732; U.S. Pat. No. 5,398,670; and U.S. Pat. No. 5,454,364. The known systems employing balloons suffer from the disadvantage that the friction afforded against the intestine's inner wall is too little to provide a reliable propulsion in said intestine, unless such balloons are inflated to such extend that they cause pain to the concerning person.

To address this problem it is also known to employ means to vacuumize against the intestine's inner wall such as known from U.S. Pat. No. 5,906,591 and U.S. Pat. No. 6,309,346.

Other systems to provide traction against the intestine's inner wall are known from U.S. Pat. No. 6,648,814 using wheels and the application of belts for this purpose is known from U.S. Pat. No. 6,695,771; U.S. Pat. No. 5,562,601; U.S. Pat. No. 6,071,234; and U.S. Pat. No. 6,224,544.

From the above discussion it is clear that there is a long felt need to provide a propulsion-unit to reliably provide traction in the intestines of a person or animal, whereby a swift operation of the unit is desirable. A condition at all times is that damage to the intestines is avoided. Such a reliable propulsion-unit is particularly needed when it is intended to move a colonoscope into a person's or animal's colon so as to inspect said colon. The problem that arises when moving a colonoscope in the colon is that the colon is not straight but shows unexpected turns, which makes it hard to move the colonoscope through the large intestine. At times this may result in the colonoscope getting stuck or damaging the intestine whereby the person or animal that undergoes the inspection may experience severe pain.

### BRIEF SUMMARY OF THE INVENTION

The invention is therefore aimed at providing a propulsion-unit which at least in part addresses the above problems and allows for application in combination with a colonoscope or with any other instrument that is required to be propelled, and in general to provide an alternative to existing systems that serve this purpose.

### BRIEF DESCRIPTION OF THE SEVERAL VIEWS OF THE DRAWINGS

The invention will hereinafter be further elucidated with reference to some exemplary embodiments of the propulsion-unit and the robot of the invention and with reference to the drawing. In the drawings:

FIG. 1 shows a first embodiment of the propulsion-unit of the invention;

FIG. 2 shows dismounted parts of the propulsion-unit shown in FIG. 1;

FIG. 3 shows a second embodiment of the propulsion-unit of the invention;

FIG. 4 shows interior parts of the propulsion-unit according to FIG. 3 with the housing of the propulsion-unit removed;

FIG. 5 shows a third embodiment of the propulsion-unit of the invention, whereby the housing of the propulsion-unit is partially removed;

FIG. 6 shows a robot comprising some propulsion-units in accordance with the invention;

FIG. 7 and FIG. 8 show two further embodiments of the propulsion-unit of the invention; and

FIG. 9 shows still a further embodiment of the propulsion-unit of the invention.

Wherever in the figures the same reference numerals are applied, these numerals refer to the same parts.

### DETAILED DESCRIPTION OF THE INVENTION

The propulsion-unit and, if applicable, any robot that is provided with such a propulsion-unit is to this end characterized by one or more of the appended claims.

In general, the propulsion-unit of the invention is characterized in that the element that is provided with the external adhesive layer is a supporting layer that supports said adhesive layer and that drive means are provided for moving the supporting layer and the body of the propulsion-unit with respect to each other. By this measure it is secured that the supporting layer with the adhesive layer provides the required frictional contact with the object along which the unit is intended to be moved. This is particularly useful as indicated above in the intestinal tract, in particular the colon of a person or animal in which the conditions are such that frictional contact with the wall of the large intestine is difficult to attain.

In order to be able to move the propulsion-unit over a sufficient length, it is desirable that the propulsion-unit is provided with at least one reel for storing the supporting layer.

There may be a reel for storing supporting layer that is being discharged such that in an application wherein the intestinal tract of a person or animal is investigated, the discharged supporting layer is left in the intestinal tract, thereby wasting its environment. It is, however, preferred that there is also a reel for winding up of the sup-porting layer during operation of the drive means for propelling the unit through the intestinal tract.

The inventors have found that the length of supporting layer that may be stored on a reel may be increased by embodying the reel or reels for the supporting layer such that it has a bearing allowing it to rotate at the body's circumference, as opposed to a construction in which the reel operates in line with the direction of movement of the propulsion-unit. This provides ample storing facility of the supporting layer, which makes the propulsion-unit of the invention very suitable for colonoscopic inspections.

Suitably the propulsion-unit is provided with at least one set of reels, each set comprising a discharge reel and a wind-up reel for a supporting layer. Advantageously, there may be several sets of reels oriented at regular positions around a longitudinal body axis of the propulsion-unit so as to provide evenly distributed forces aimed at the inner wall of the intestines. Each of the (discharge and wind-up) reels may also carry more than one supporting layer, in order to meet desired length and width or diameter requirements of the propulsion-unit.

Suitably further the drive means provide, while the unit is in use, traction to the supporting layer so as to have this supporting layer move as compared to the body of the propulsion-unit.

Preferably, the drive means are drivingly connected to the reel or reels for winding up the supporting layer.

Advantageously the drive means are arranged for selectively driving the respective supporting layers that may be provided at the outer circumference of the unit, at individually defined speeds. This allows that the propulsion-unit has a controllable direction of movement by properly controlling the speeds of said supporting layers.

A preferable embodiment of the propulsion-unit of the invention has the feature that it comprises a housing that is provided with at least one slit through which the supporting layer is guided and that the supporting layer lies adjacent to the housing, or adjacent to parts fixed to the housing.

A very practical embodiment in which the supporting layer after its use is prevented from wasting its environment is embodied with the feature that same has a housing that is provided with at least one frontal slit and at least one backward slit, and that the supporting layer is guided from within the housing through the frontal slit and adjacent to the housing to the backward slit and through said backward slit back into the housing.

It is preferable, particularly in the just-mentioned embodiment, that the at least one reel or the reels of a set of reels for storing the supporting layer or layers is or are located near to the at least one slit or slits in the housing.

The propulsion-unit of the invention may suitably be embodied such that the body equates with the housing. If this is the case, usually the reels for storing the supporting layer or layers release and take up the supporting layer or layers straightly in line with the propulsion direction of the propulsion-unit of the invention.

A most preferred embodiment of the propulsion-unit of the invention is however characterized in that the body is provided in a housing that has frontal and backward slits through which a supporting layer or supporting layers are guided adjacent to the housing, and which supporting layer or supporting layers are in use moved from within the housing through the frontal slit or slits towards the backward slit or slits and back into the housing. Particularly, with this embodiment it is possible to store large amounts of supporting layers, which facilitates the movement of the propulsion-unit over extended length in the intestinal tract, or in the other application areas in which the propulsion-unit may be employed.

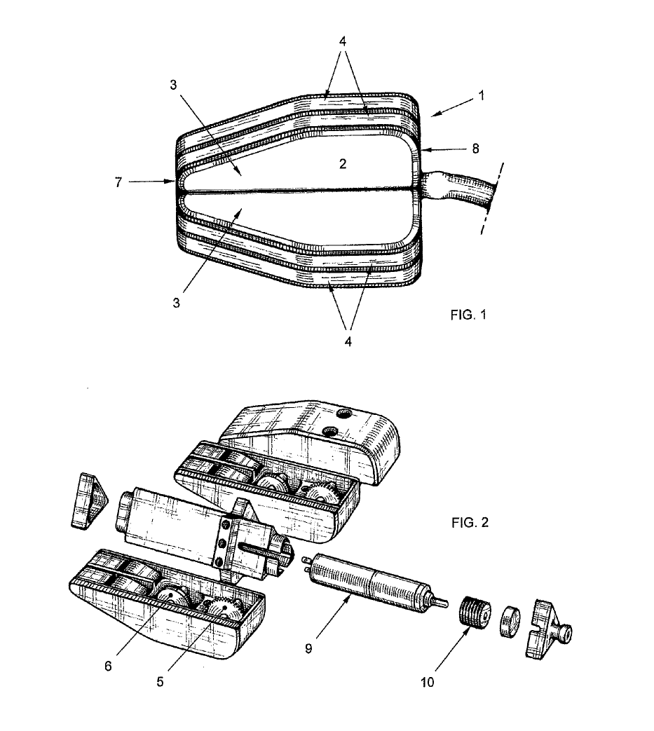

FIG. 1 shows a first embodiment of the propulsion-unit 1 of the invention having a body 2, which also acts as the unit's housing.

The propulsion-unit 1 is embodied with several parts 3 at the outer circumference of which, a film 4 is guided. In this embodiment the supporting layer is a film 4 that supports an adhesive layer facing away from the parts 3 so as to be able to come in contact with a surface of an object along which the unit 1 has to be propelled. Depending on the circumstances instead of a film 4, also use can be made of a caterpillar track, a belt or any other suitable means to support the adhesive layer.

The unit 1 is further provided with drive means 9, 10, part of which is shown in FIG. 2 showing several lose components of the propulsion-unit 1 of FIG. 1.

It will be clear for the artisan that the film 4 is moved from a reel 6 on the inside of the body 2 through a slit at the frontal end 7 (see FIG. 1) of the unit 1 over and adjacent to the housing 2 towards the back end 8 of the unit 1 so as to be wound up by a further reel 5, which is driven by a preferably electric motor 9 that connects to said reel 5 through a worm wheel 10. When there are more films movable at the outer circumference of the housing 2, there may also be a dedicated electric motor for each film 4, which gives the possibility that each film 4 is having a selectively controllable speed for controlling the movement direction of the unit 1.

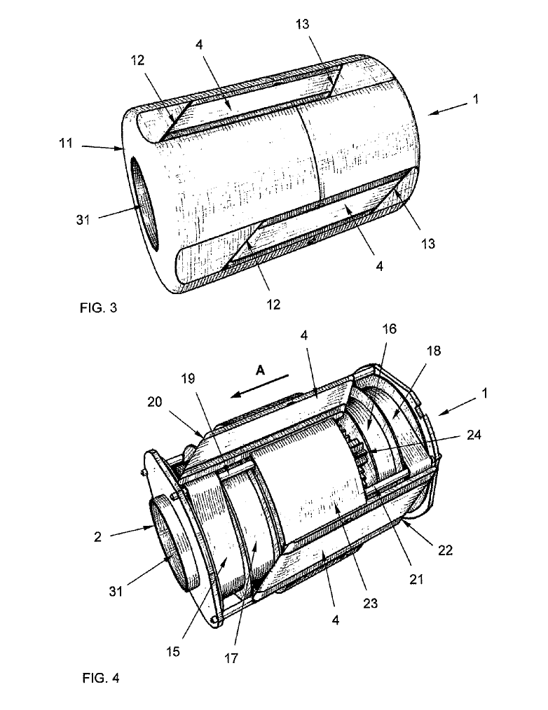

FIG. 3 shows a second embodiment of the propulsion-unit 1 of the invention which is embodied with a housing 11 that is provided with at least one slit 12, 13 through which the film 4 is guided, wherein the film 4 lies adjacent to the housing 11.

FIG. 4 clearly shows that the unit 1 has at least one, and in the shown case two sets of reels 15, 16; 17, 18, each set comprising a discharge reel 15, 17 and a wind-up reel 16, 18 for film 4. Also this FIG. 4 shows clearly that the reels 15, 16; 17, 18 for the film 4 have a bearing allowing it to rotate at the circumference of the body 2. This construction in which the reels 15, 16; 17, 18 are bearingly mounted on the body 2 so as to be able to rotate around said body's circumference for releasing the film 4 from the reels 15, 17 and winding up the film 4 on the reels 16, 18, in a direction perpendicular to the movement direction of the body 2 as indicated with arrow A, arranges that the film 4 may be stored in large quantities on the respective reels 15, 16; 17, 18. Said reels may be arranged to store several films, each of the films on a particular reel leaving and entering the housing through a different slit. The drive means for providing traction to these films may be individually controllable to provide the propulsion-unit with steering capability.

Further there are guiding bars 19, 20, 21, and 22 for guiding the film 4 from the earlier-mentioned perpendicular direction to a direction parallel to the movement direction A of the body 2, such that the films come to lie adjacent to the outer circumference of the housing 11. Thus, it is possible that the film is guided from the discharge reels 15, 17 within the housing 11 through the frontal slits 12 in the housing 11 and guided adjacent to said housing 11 to the backward slits 13 and through said backward slits 13 back into the housing 11 so as to be wound up on the wind-up reels 16, 18 that are provided in the housing 11. Suitably, for this purpose the reels 15, 17 from which the films 4 are discharged, are located near the frontal slits 12 whereas the wind-up reels 16, 18 for winding up the used films are located near to the backward slits 13 in the housing 11.

At a central part 23 the body 2 of the propulsion-unit 1 is provided with drive means for driving the film or films 4. For this purpose for instance a transmission gear connecting the wind-up reels 16, 18 to a motor or motors, or other means of driving said wind-up reels 16, 18 may be employed. Said transmission gear forming part of the drive means is indicated with reference numeral 24.

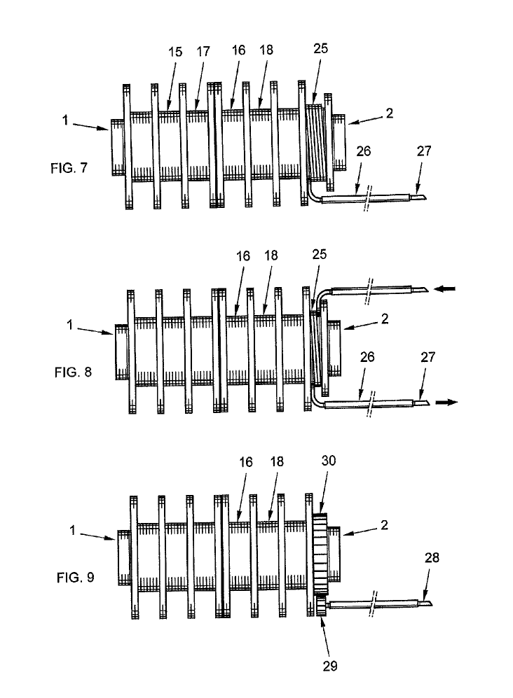

FIG. 7 shows an embodiment of the propulsion-unit 1 of the invention, in which the drive means comprise a pulling cable 25 that is provided in the housing and wound around a suitable part of the body 2 of the propulsion-unit. For clarity purposes the housing is not shown in FIG. 7. The pulling cable 25 connects to wind-up reels 16, 18 either directly or via a transmission gear, and said pulling cable 25 is further guided out of the (not shown) housing through a Bowden-cable 26 to a position 27 distant from the propulsion-unit 1 and its housing so as to be operated therefrom. Instead of a Bowden-cable other suitable guide means that are flexible and axially incompressible can be employed as well, such as a hose, a catheter, a pulling spring. These means may also be employed in plural, as well as there may be plural pulling cables. In the case of plural pulling cables, each cable 25 may connect to a separate wind-up reel for providing individual traction to a film 4, such that the films 4 collectively determine the movement direction of the unit 1.

An embodiment comparable to what is shown in FIG. 7 is shown in FIG. 8. This embodiment comprises either one pulling cable 25 having two free extensions, or two pulling cables 25 making possible that the wind-up reel 16 can be driven in two opposite directions.

Still a further alternative is shown in FIG. 9, in which again the housing is not shown for clarity purposes. In this embodiment the drive means comprise a flexible shaft 28 having a transmission gear 29 near to a corresponding gear 30 on the body 2 that connects to the wind-up reels 16, 18. By rotating the shaft 28 it is possible to wind up the (not shown) film on the wind-up reels 16, 18 and thus provide traction to the propulsion-unit 1 by having the film 4 move along the housing's outer circumference in the longitudinal direction of the unit 1.

Instead of said flexible shaft it is also possible to use a chain of universal joints, a torsionally stiff tube or braided tube or in general a long, flexible torsionally rigid driving body which is rotatable around its body-axis.

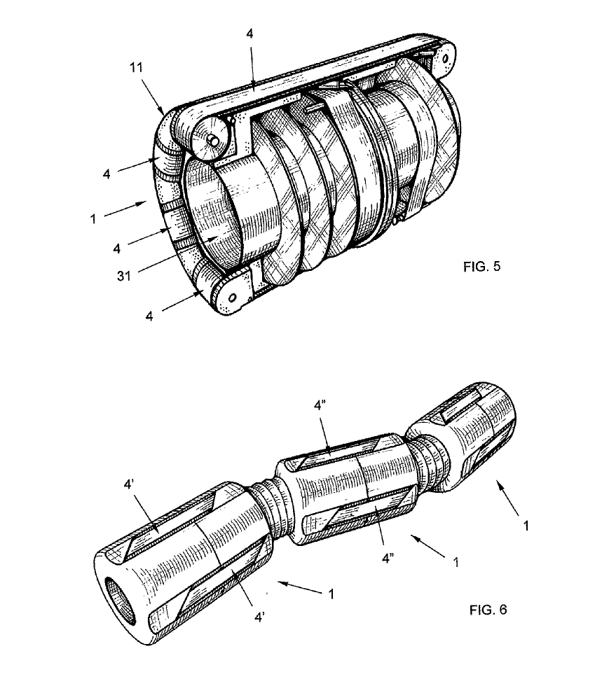

With reference to FIG. 5 a further embodiment of the propulsion-unit 1 of the invention is shown, in which the film or films 4 are guided from a front side to a back side of the housing 11, thereby a larger part of the outer circumference of the housing 11 resulting in improved traction along the inner wall of the object along which the unit 1 is moved.

Finally, FIG. 6 shows a combination of propulsion-units 1 embodied with films 4′ and 4″ that are moveable in opposite directions. In this way the robot as shown in FIG. 6 when provided with its own power unit can simply move preprogrammed or with remote control in either direction.

It is further remarked that the propulsion-unit of the invention may be varied in many respects without departing from the gist of the invention as embodied in the appended claims as for instance is illustrated by the embodiment shown in FIG. 5.

The appended claims are therefore not to be considered restricted by the shown embodiments which only serve to resolve any possible ambiguities in respect of the claims.

Amongst the variations that are possible without departing from the scope of protection that merits the invention, the propulsion-unit 1 of the invention may be embodied with a support for the films 4 at the outer circumference of the housing 11 in the form of pressing means for pressing said films away from the housing 11. This may greatly improve the frictional contact between the films 4 of the propulsion-unit with the inner walls of the object's surface along which the unit moves. The pressing means are also beneficial if the propulsion-unit moves in a tube having a varying diameter.

Furthermore, particularly FIGS. 3-6 show that the unit 1 may be provided with a central channel 31 through the body 2 for received therein an instrument used for inspecting or treating such as a camera or even a colonoscope, or any other instrument that is considered useful. This instrument may for instance concern a biopsy channel, one or more light sources, one or more analog or digital camera's (preferably remote-controlled), ultrasound probes, or other diagnostic tools, grasping forceps, scissors, biopsy forceps, or other tools for tissue handling and manipulations, a flexible endoscope like a colonoscope, an enteroscope, a sigmoidoscope, a gastroscope, a duodenoscope, a cystoscope, an arthroscope, a larynchoscope, an ureteroscope, a bronchoscope, a fiberscope, or a catheter, a guidewire, a sending and receiving unit for wireless control, a power supply for wireless control, an industrial flexible endoscope such as a boroscope or a video-scope. The propulsion-unit may also effectively be used in combination with an overtube covering one of the above mentioned instruments. It is further remarked that the film or films 4 that carries the adhesive layer can be suitably provided at regular distances with portions having hydrophilic properties, such as dry paper. This affords the advantage that when the adhesive layer is a muco-adhesive layer which is very suited for sticking to the wall of the intestines, it is possible to control the humidity condition at which the layer operates. In this manner it is possible to deal with circumstances that may otherwise deteriorate the adhesive properties of the muco-adhesive layer.

The muco-adhesive properties of the films can further be promoted by application of holes in the film or films 4 or by providing said films with specific micropatterns.

When the adhesive layer is a muco-adhesive layer, it is further possible to provide the propulsion-unit of the invention with means to release or control the humidity conditions near to the muco-adhesive layer. By these means—for instance water supply means—it is possible to locally neutralize the adhesive properties of said layer, which affords the advantage that directional control of a unit is then possible. The water supply means have for this purpose several selectively controllable outlets near to the muco-adhesive layers which are distributed around the outer circumference of the propulsion-unit.

What is claimed is:

1. Propulsion-unit provided with a body and comprising at least one element having an external adhesive layer for providing frictional contact with an object's surface along which the unit in use moves, wherein the element is a supporting layer that supports said adhesive layer, and wherein drive means are provided for moving the supporting layer and the body with respect to each other.

2. Propulsion-unit according to claim 1, wherein same is provided with at least one reel for storing the supporting layer.

3. Propulsion-unit according to claim 1, wherein the body houses or supports at least one reel for discharging and/or winding up of the supporting layer during operation of the drive means.

4. Propulsion-unit according to claim 1, wherein at least one reel for the supporting layer is provided which reel has a bearing allowing it to rotate at the body's circumference.

5. Propulsion-unit according to claim 1, wherein same is provided with at least one set of reels, each set comprising a discharge reel and a wind-up reel for the supporting layer.

6. Propulsion-unit according to claim 1, wherein the drive means provide in use traction to the supporting layer.

7. Propulsion-unit according to claim 1, wherein the drive means drive in use at least one reel for winding up the supporting layer.

8. Propulsion-unit according to claim 1, wherein same has a housing that is provided with at least one slit through which the supporting layer is guided, and wherein said supporting layer lies adjacent to the housing.

9. Propulsion-unit according to claim 1, wherein same has a housing in which at least one reel for the supporting layer is housed, which supporting layer is guided through at least one slit in the housing and adjacent to its outside surface from a frontal portion to a back position of the housing.

10. Propulsion-unit according to claim 1, wherein same has a housing that is provided with at least one frontal slit and at least one backward slit, and that the supporting layer is guided from within the housing through the frontal slit and adjacent to the housing to the back-ward slit and through said backward slit back into the housing.

11. Propulsion-unit according to claim 9 wherein at least one reel of a set of reels for storing the supporting layer is or are located near to the at least one slit or slits in the housing.

12. Propulsion-unit according to claim 8, wherein the body is the housing.

13. Propulsion-unit according to claim 10, wherein the body is provided in a housing that has frontal and backward slits through which a supporting layer or supporting layers are guided adjacent to the housing, and which supporting layer or supporting layers are in use moved from within the housing through the frontal slit or slits towards the backward slit or slits and back into the housing.

14. Propulsion-unit provided with a housing and/or a body supporting at least one element having an external adhesive layer for providing frictional contact with an object's surface along which the unit in use moves, wherein the element is a supporting layer that supports said adhesive layer, and wherein the supporting layer is movable between frontal and backward slits in the housing and at the housing's outer circumference.

15. Propulsion-unit according to claim 14, wherein the body supports a first reel or reels for storing and unwinding the supporting layer or supporting layers, which supporting layer or supporting layers are guided from within the housing through a frontal slit or slits to the housing's outside and which supporting layer or supporting layers lie adjacent to said housing and are guided through a backward slit or slits to a second reel or reels supported by the body for winding up the supporting layer or supporting layers.

16. Propulsion-unit according to claim 14, wherein it is provided with drive means for driving the supporting layer or supporting layers.

17. Propulsion-unit according to claim 14, wherein it is provided with drive means for driving a reel or reels for the supporting layer or supporting layers.

18. Propulsion-unit according to claim 17, wherein there are at least two supporting layers and that the drive means are arranged for selectively driving the supporting layers at individually defined speeds.

19. Propulsion-unit according to claim 17, wherein the drive means are supported by the body and/or the housing.

20. Propulsion-unit according to claim 17, wherein the drive means comprises an electrical motor or motors.

21. Propulsion-unit according to claim 17, wherein the drive means comprise at least one pulling cable provided in the housing and guided from said housing through a flexible and axially incompressible guide tube to a position distant from the housing which guide tube is selected from the group consisting of a Bowden-cable, a hose, a catheter, a pulling spring.

22. Propulsion-unit according to claim 17, wherein the drive means are selected from the group comprising a flexible shaft, a chain of universal joints, a torsionally stiff tube or braided tube, a long and flexible torsionally rigid driving body for imparting a torsional driving torque to a reel or reels for the supporting layer or supporting layers.

23. Propulsion-unit according to claim 15, wherein the reel or reels are bearingly mounted on the body so as to be able to rotate around said body's circumference for discharging and/or winding up the supporting layer or supporting layers in a direction perpendicular to the movement direction (A) of the body, and wherein there are guiding bars for guiding said supporting layer or supporting layers from said perpendicular direction to a direction parallel to the movement direction (A) of the body wherein the supporting layer or supporting layers lie adjacent to the housing's outer circumference.

24. Propulsion-unit according to claim 14, wherein the supporting layer or supporting layers are supported by pressing means for pressing said supporting layer or supporting layers away from said housing.

25. Propulsion-unit according to claim 14, wherein it is provided with a central channel through said body for receiving therein an instrument selected from the group comprising a biopsy channel, one or more light sources, one or more analog or digital camera's, ultrasound probes or other diagnostic tools, grasping forceps, scissors, biopsy forceps, or other tissue handling or manipulation tools, a flexible endoscope like a colonoscope, an enteroscope, a sigmoidoscope, a gas-troscope, a duodenoscope, a cystoscope, an arthroscope, a larynchoscope, an ureteroscope, a bronchoscope, a fiberscope, or a catheter, a guidewire, a sending and receiving unit for wireless control, a power supply for wireless control, an industrial flexible endoscope such as a baroscope or a video-scope.

26. Propulsion-unit according to claim 14, wherein the adhesive layer provided on the supporting layer or supporting layers is a muco-adhesive layer.

27. Propulsion-unit according to claim 14, wherein the supporting layer or supporting layers carrying the adhesive layer is provided at regular distances with portions having hydrophilic properties.

28. Propulsion-unit according to claim 14, wherein the supporting layer is selected from the group comprising a film, a caterpillar track, and a belt.

29. Propulsion-unit according to claim 26, wherein there are water supply means to neutralize the adhesive properties of the muco-adhesive layer.

30. Propulsion-unit according to claim 29, wherein the water supply means are selectively controllable for providing directional control to the unit.

31. Robot provided with at least one propulsion-unit according to claim 1.

32. Robot according to claim 31, wherein it comprises at least two propulsion-units, and wherein the at least two propulsion-units have supporting layers that are moveable in opposite directions.
